SEQUENCE LISTING (4 sequences)

SEQ ID NO: 1 - protein, 58 aa, Staphylococcus aureus [sequence omitted]

SEQ ID NO: 2 - protein, 58 aa, Artificial Sequence [sequence omitted]

SEQ ID NO: 3 - protein, 63 aa, Artificial Sequence
  synthetic peptide
AQGTVDAKFDKEQQNAFYEILHLPNLTEEQRNAFIQSLKDDPSQSANLLAEAKKLNDAQAPKC

SEQ ID NO: 4 - protein, 122 aa, Artificial Sequence
  synthetic peptide
AQGTVDAAKFDKEQQNAFYEILHLPNLTEEQRNAFIQSLKDDPSQSANLLAEAKKLNDAQAPKVDAKFDKEQQNAFYEILHLPNLTEEQRNAFIQSLKDDPSQSANLLAEAKKLNDAQAPKC

(19) United States
(12) Patent Application Publication
(10) Pub. No.: US 2012/0149875 A1
(43) Pub. Date: Jun. 14, 2012

(54) AFFINITY CHROMATOGRAPHY MATRIX

(75) Inventors: Hans J. Johansson, Uppsala (SE); Anders Ljunglof, Uppsala (SE); Ronnie Palmgren, Stockholm (SE)

(73) Assignee: GE HEALTHCARE BIO-SCIENCES AB, UPPSALA (SE)

(21) Appl. No.: 13/143,996

(22) PCT Filed: Jan. 11, 2010
(86) PCT No.: PCT/SE2010/050016

§ 371 Date: Sep. 26, 2011

(30) Foreign Application Priority Data
Jan. 12, 2009 (SE) 0900029-0

(51) Int. Cl. C07K 1/22; C07K 19/00; C07K 16/00

(52) U.S. Cl. 530/387.3; 530/387.1; 530/388.1

(57) ABSTRACT

The present invention relates to a method of separating one or more immunoglobulin containing proteins from a liquid. The method includes first contacting the liquid with a separation matrix comprising ligands immobilised to a support; allowing the immunoglobulin containing proteins to adsorb to the matrix by interaction with the ligands; followed by an optional step of washing the adsorbed immunoglobulin containing proteins; and recovering said immunoglobulin containing proteins by contacting the matrix with an eluent which releases the proteins. The method improves upon previous separation methods being that each of the ligands consists essentially of a monomer or dimer SpA or protein Z or a functional variant thereof.

### FIELD OF THE INVENTION

The present invention relates to the field of affinity chromatography, and more specifically to separation matrix containing ligand monomers or dimers. The invention also relates to methods for the separation of proteins of interest with aforementioned matrix, with the advantage of increased capacity and elution pH.

### BACKGROUND OF THE INVENTION

Immunoglobulins represent the most prevalent biopharmaceutical products in either manufacture or development by organisations worldwide. The high commercial demand for and hence value of this particular therapeutic market has lead to the emphasis being placed on pharmaceutical companies to maximise the productivity of their respective mAb manufacturing processes whilst controlling the associated costs.

Affinity chromatography is used in most cases, as one of the key steps in the purification of these immunoglobulin molecules, such as monoclonal or polyclonal antibodies. A particularly interesting class of affinity reagents is proteins capable of specific binding to invariable parts of an immunoglobulin molecule, such interaction being independent on the antigen-binding specificity of the antibody. Such reagents can be widely used for affinity chromatography recovery of immunoglobulins from different samples such as but not limited to serum or plasma preparations or cell culture derived feed stocks. An example of such a protein is staphylococcal protein A, containing domains capable of binding to the Fc and Fab portions of IgG immunoglobulins from different species.

Staphylococcal protein A (SpA) based reagents have due to their high affinity and selectivity found a widespread use in the field of biotechnology, e.g. in affinity chromatography for capture and purification of antibodies as well as for detection. At present, SpA-based affinity medium probably is the most widely used affinity medium for isolation of monoclonal antibodies and their fragments from different samples including industrial feed stocks from cell cultures. Accordingly, various matrices comprising protein A-ligands are commercially available, for example, in the form of native protein A (e.g. Protein A SEPHAROSE™, GE Healthcare, Uppsala, Sweden) and also comprised of recombinant protein A (e.g. rProtein A SEPHAROSE™, GE Healthcare). More specifically, the genetic manipulation performed in the commercial recombinant protein A product is aimed at facilitating the attachment thereof to a support.

These applications, like other affinity chromatography applications, require comprehensive attention to definite removal of contaminants. Such contaminants can for example be non-eluted molecules adsorbed to the stationary phase or matrix in a chromatographic procedure, such as non-desired biomolecules or microorganisms, including for example proteins, carbohydrates, lipids, bacteria and viruses. The removal of such contaminants from the matrix is usually performed after a first elution of the desired product in order to regenerate the matrix before subsequent use. Such removal usually involves a procedure known as cleaning-in-place (CIP), wherein agents capable of eluting contaminants from the stationary phase are used. One such class of agents often used is alkaline solutions that are passed over said stationary phase. At present the most extensively used cleaning and sanitising agent is NaOH, and the concentration thereof can range from 0.1 up to e.g. 1 M, depending on the degree and nature of contamination. This strategy is associated with exposing the matrix for pH-values above 13. For many affinity chromatography matrices containing proteinaceous affinity ligands such alkaline environment is a very harsh condition and consequently results in decreased capacities owing to instability of the ligand to the high pH involved.

An extensive research has therefore been focussed on the development of engineered protein ligands that exhibit an improved capacity to withstand alkaline pH-values. For example, Gülich et al (Susanne Gülich, Martin Linhult, Per-Åke Nygren, Mathias Uhlén, Sophia Hober, Journal of Biotechnology 80 (2000), 169-178) suggested protein engineering to improve the stability properties of a Streptococcal albumin-binding domain (ABD) in alkaline environments. Gülich et al created a mutant of ABD, wherein all the four aspargine residues have been replaced by leucine (one residue), asparte (two residues) and lysine (one residue). Further, Gülich et al report that their mutant exhibits a target protein binding behaviour similar to that of the native protein, and that affinity columns containing the engineered ligand show higher binding capacities after repeated exposure to alkaline conditions than columns prepared using the parental non-engineered ligand. Thus, it is concluded therein that all four asparagine residues can be replaced without any significant effect on structure and function.

Recent work show that changes can also be made to protein A (SpA) to effect similar properties. US patent application publication 2005/0143566 discloses that when at least one asparagine residue is mutated to an amino acid other than glutamine or aspartic acid, the mutation confers an increased chemical stability at pH-values of up to about 13-14 compared to the parental SpA, such as the B-domain of SpA, or Protein Z, a synthetic construct derived from the B-domain of SpA (U.S. Pat. No. 5,143,844). The authors show that when these mutated proteins are used as affinity ligands, the separation media as expected can better withstand cleaning procedures using alkaline agents. US 2006/0194955 shows that the mutated ligands can better withstand proteases thus reducing ligand leakage in the separation process. Another publication, US 2006/0194950 shows that the alkali stable SpA domains can be further modified such that the ligands lacks affinity for Fab but retains Fc affinity, for example by a G29A mutation.

Historically the native protein A containing 5 IgG binding domains was used for production of all protein A affinity media. Using recomenband technology a number of protein A construct have been produced all containing 4 or 5 IgG binding domains.

Accordingly, there is a need in this field to obtain a separation matrix containing protein ligands having a lower number of repeats yet with similar or increased binding capacity as the tetramers.

### BRIEF SUMMARY OF THE INVENTION

One object of the present invention is to provide an affinity separation matrix, which comprises protein ligands capable of binding immunoglobulins, such as IgG, IgA and/or IgM, preferably via their Fc-fragments. These ligands are presented as monomers or dimers and have a higher relative binding capacity, as compared to ligand multimers with a higher number of repeat, e.g. pentameric ligands.

Another object of the invention is to provide a method for separating one or more immunoglobulin containing proteins, using the current affinity matrix. By using a monomer or dimeric affinity ligand the method unexpectedly achieves increased relative binding capacity for the target molecules. Further, using a monomeric ligand, the elution pH increases.

Thus the invention provides a method for either producing a purified product, such as a pure immunoglobulin fraction or alternatively a liquid from which the immunoglobulin has been removed, or to detect the presence of immunoglobulin in a sample. The ligands according to the invention exhibit an increased capacity, which renders the ligands attractive candidates for cost-effective large-scale operation.

One or more of the above-defined objects can be achieved as described in the appended claims.

### BRIEF DESCRIPTION OF THE DRAWINGS

FIG. 1 shows breakthrough curves recorded at 6 minutes residence time for Z1 (broken line), Z 2 (dotted line) and Z 4 (unbroken line).

FIG. 2: analytical size exclusion chromatography on Superdex 200 5/150 GL. Fc-Fusion Protein (unbroken line) and MAb 3 (dotted line).

FIG. 3: elution pH of different MAbs and Fc-fusion protein, low load, applied on various z-prototypes.

### DETAILED DESCRIPTION OF THE INVENTION

### Definitions

The term “protein” is used herein to describe proteins as well as fragments thereof. Thus, any chain of amino acids that exhibits a three dimensional structure is included in the term “protein”, and protein fragments are accordingly embraced.

The term “functional variant” of a protein means herein a variant protein, wherein the function, in relation to the invention defined as affinity and stability, are essentially retained. Thus, one or more amino acids those are not relevant for said function may have been exchanged.

The term “parental molecule” is used herein for the corresponding protein in the form before a mutation according to the invention has been introduced.

The term “structural stability” refers to the integrity of three-dimensional form of a molecule, while “chemical stability” refers to the ability to withstand chemical degradation.

The term “Fc fragment-binding” protein means that the protein is capable of binding to the Fc fragment of an immunoglobulin. However, it is not excluded that an Fc fragment-binding protein also can bind other regions, such as Fab regions of immunoglobulins.

In the present specification, if not referred to by their full names, amino acids are denoted with the conventional one-letter symbols.

Mutations are defined herein by the number of the position exchanged, preceded by the wild type or non-mutated amino acid and followed by the mutated amino acid. Thus, for example, the mutation of an asparagine in position 23 to a threonine is denoted N23T.

The present invention in one aspect relates to a method of separating one or more immunoglobulin containing proteins from a liquid, which method comprises (a) contacting the liquid with a separation matrix comprising ligands immobilised to a support; (b) allowing the immunoglobulin containing proteins to adsorb to the matrix by interaction with the ligands; (c) an optional step of washing the adsorbed immunoglobulin containing proteins; and (d) recovering the immunoglobulin containing proteins by contacting the matrix with an eluent which releases the proteins. The method provides increased binding capacity of the ligands to the immunoglobulin molecules by using a monomeric ligand, such as domain B of staphylococcal Protein A (SpA) or protein Z.

In another aspect the invention relates to a method of separating one or more immunoglobulin containing proteins from a liquid, which method comprises (a) contacting the liquid with a separation matrix comprising ligands immobilised to a support; (b) allowing immunoglobulin containing proteins to adsorb to the matrix by interaction with the ligands; (c) an optional step of washing the adsorbed immunoglobulin containing proteins; and (d) recovering the immunoglobulin containing proteins by contacting the matrix with an eluent which releases the proteins. The method provides increased binding capacity of the ligands to the immunoglobulin molecules by using a dimeric ligand, such as a domain B of staphylococcal Protein A (SpA) or protein Z.

The immunoglobulin-binding protein (i.e., ligand) can be any protein with a native immunoglobulin-binding capability, such as Staphylococcal protein A (SpA) or Streptococcal protein G (SpG). For a review of other such proteins, see e.g. Kronvall, G., Jonsson, K. Receptins: a novel term for an expanding spectrum of natural and engineered microbial proteins with binding properties for mammalian proteins, J. Mol. Recognit. 1999 January-February; 12(1):38-44. The monomeric or dimeric ligands can comprise one of more of the E, D, A, B and C domains of SpA. More preferably the ligands comprise domain B of protein A or the engineered protein Z.

In one embodiment, the ligands are rendered alkali-stable, such as by mutating at least one asparagine residue of the SpA domain B or protein Z to an amino acid other than glutamine. As discussed earlier, US patent application publication 2005/0143566 discloses that when at least one asparagine residue is mutated to an amino acid other than glutamine or aspartic acid, the mutation confers an increased chemical stability at high pH. Further, affinity media including these ligands can better withstand cleaning procedures using alkaline agents. US 2006/0194955 shows that the mutated ligands can also better withstand proteases thus reducing ligand leakage in the separation process. The disclosures of these applications are hereby incorporated by reference in their entirety.

In another embodiment, the ligand(s) so prepared lack any substantial affinity for the Fab part of an antibody, while having affinity for the Fc part. In certain embodiments, at least one glycine of the ligands has been replaced by an alanine. US 2006/0194950 shows that the alkali stable domains can be further modified such that the ligands lacks affinity for Fab but retains Fc affinity, for example by a G29A mutation. The disclosure of the application is hereby incorporated by reference in its entirety. The numbering used herein of the amino acids is the conventionally used in this field, and the skilled person in this field can easily recognize the position to be mutated.

In an advantageous embodiment, the alkali-stability of domain B has been achieved by mutating at least one asparagine residue to an amino acid other than glutamine; and contains a mutation of the amino acid residue at position 29 of the alkali-stable domain B, such as a G29A mutation.

In another embodiment, the ligand is Protein Z in which the alkali-stability has been achieved by mutating at least one asparagine residue to an amino acid other than glutamine. In an advantageous embodiment, the alkali-stability has been achieved by mutating at least the asparagine residue at position 23 to an amino acid other than glutamine. In another embodiment, the alkali-stable protein is a native protein which is substantially stable at alkaline conditions.

As the skilled person in this field will easily understand, the mutations to provide alkaline-stability and the G to A mutation may be carried out in any order of sequence using conventional molecular biology techniques. Further, the ligands can be expressed by a vector containing a nucleic acid sequence encoding the mutated protein ligands. Alternatively, they can also be made by protein synthesis techniques. Methods for synthesizing peptides and proteins of predetermined sequences are well known and commonly available in this field.

Thus, in the present invention, the term “alkali-stable domain B of Staphylococcal Protein A” means an alkali-stabilized protein based on Domain B of SpA, such as the mutant protein described in US patent application publication 2005/0143566 and US 2006/0194950; as well as other alkali-stable proteins of other origin but having a functionally equivalent amino acid sequence.

As the skilled person will understand, the expressed protein should be purified to an appropriate extent before being immobilized to a support. Such purification methods are well known in the field, and the immobilization of protein-based ligands to supports is easily carried out using standard methods. Suitable methods and supports will be discussed below in more detail.

Accordingly, in one embodiment, a mutated protein according to the invention comprises at least about 75%, such as at least about 80% or preferably at least about 95%, of the sequence as defined in SEQ ID NOs: 1 or 2, with the proviso that the asparagine mutation is not in position 21.

In the present specification, SEQ ID NO: 1 defines the amino acid sequence of the B-domain of SpA:

Ala Asp Asn Lys Phe Asn Lys Glu Gln Gln Asn Ala Phe Tyr Glu Ile
Leu His Leu Pro Asn Leu Asn Glu Glu Gln Arg Asn Gly Phe Ile Gln
Ser Leu Lys Asp Asp Pro Ser Gln Ser Ala Asn Leu Leu Ala Glu Ala
Lys Lys Leu Asn Asp Ala Gln Ala Pro Lys.

SEQ ID NO: 2 defines a protein known as protein Z:

Val Asp Asn Lys Phe Asn Lys Glu Gln Gln Asn Ala Phe Tyr Glu Ile
Leu His Leu Pro Asn Leu Asn Glu Glu Gln Arg Asn Ala Phe Ile Gln
Ser Leu Lys Asp Asp Pro Ser Gln Ser Ala Asn Leu Leu Ala Glu Ala
Lys Lys Leu Asn Asp Ala Gln Ala Pro Lys.

Protein Z is a synthetic construct derived from the B-domain of SpA, wherein the glycine in position 29 has been exchanged for alanine, see e.g. Ståhl et al, 1999: Affinity fusions in biotechnology: focus on protein A and protein G, in The Encyclopedia of Bioprocess Technology: Fermentation, Biocatalysis and Bioseparation. M. C. Fleckinger and S. W. Drew, editors. John Wiley and Sons Inc., New York, 8-22.

In one embodiment, the above described mutant protein is comprised of the amino acid sequence defined in SEQ ID NOs: 1 or 2, or is a functional variant thereof. The term “functional variant” as used in this context includes any similar sequence, which comprises one or more further variations in amino acid positions that have no influence on the mutant protein's affinity to immunoglobulins or its improved chemical stability in environments of increased pH-values.

In an advantageous embodiment, the present mutation(s) are selected from the group that consists of N23T; N23T and N43E; N28A; N6A; N11S; N11S and N23T; and N6A and N23T; and wherein the parental molecule comprises the sequence defined by SEQ ID NO: 2. As mentioned above, in order to achieve a mutant protein useful as a ligand with high binding capacity for a prolonged period of time in alkaline conditions, mutation of the asparagine residue in position 21 is avoided. In one embodiment, the asparagine residue in position 3 is not mutated.

In the most advantageous embodiment, an asparagine residue located between a leucine residue and a glutamine residue has been mutated, for example to a threonine residue. Thus, in one embodiment, the asparagine residue in position 23 of the sequence defined in SEQ ID NO: 2 has been mutated, for example to a threonine residue. In a specific embodiment, the asparagine residue in position 43 of the sequence defined in SEQ ID NO: 2 has also been mutated, for example to a glutamic acid. In the embodiments where amino acid number 43 has been mutated, it appears to most advantageously be combined with at least one further mutation, such as N23T.

The finding that the various asparagine residues of the B-domain of SpA and protein Z can be ascribed different contributions to affinity and stability properties of the mutated protein was quite unexpected, especially in view of the above discussed teachings of Gülich et al wherein it was concluded that all the asparagine residues of ABD could be mutated without any internal discrimination.

Thus, the invention encompasses the above-discussed monomeric mutant proteins. However, such protein monomers can be combined into multimeric proteins, such as dimers, trimers, tetramers, pentamers etc. Accordingly, another aspect of the present invention is a multimer comprised of at least one of the mutated proteins according to the invention together with one or more further units, preferably also mutant proteins according to the invention. Thus, the present invention is e.g. a dimer comprised of two repetitive units.

In one embodiment, the multimer according to the invention comprises monomer units linked by a stretch of amino acids preferably ranging from 0 to 15 amino acids, such as 5-10. The nature of such a link should preferably not destabilise the spatial conformation of the protein units. Furthermore, said link should preferably also be sufficiently stable in alkaline environments not to impair the properties of the mutated protein units.

In one embodiment, the present monomeric ligands comprise the sequence of SEQ ID NO: 3:

AlaGlnGlyThrValAspAlaLysPheAspLysGluGlnGlnAsnAla
PheTyrGluIleLeuHisLeuProAsnLeuThrGluGluGlnArgAsn
AlaPheIleGlnSerLeuLysAspAspProSerGlnSerAlaAsnLeu
LeuAlaGluAlaLysLysLeuAsnAspAlaGlnAlaProLysCys.

In another embodiment, the present dimeric ligands comprise the sequence of SEQ ID NO: 4:

AlaGlnGlyThrValAspAlaLysPheAspLysGluGlnGlnAsnAla
PheTyrGluIleLeuHisLeuProAsnLeuThrGluGluGlnArgAsn
AlaPheIleGlnSerLeuLysAspAspProSerGlnSerAlaAsnLeu
LeuAlaGluAlaLysLysLeuAsnAspAlaGlnAlaProLysValAsp
AlaLysPheAspLysGluGlnGlnAsnAlaPheTyrGluIleLeuHis
LeuProAsnLeuThrGluGluGlnArgAsnAlaPheIleGlnSerLeu
LysAspAspProSerGlnSerAlaAsnLeuLeuAlaGluAlaLysLys
LeuAsnAspAlaGlnAlaProLysCys.

The current invention unexpected found that when comparing the capacity of the ligands, while comparable high dynamic binding capacity was obtained for tetramers and dimers, the monomers has the highest relative capacity (mg MAb/mg ligand). In general it is found that higher relative capacity is obtained for ligands with fewer z-units. Our data also confirm that elution pH is dependent on ligand density. Further, the samples purified on the monomer ligand prototype elute with higher pH compared with the other prototypes, an advantage for immunoglobulins susceptible to aggregation at low pH. In the mean time, clearance of host cell proteins was almost equivalent for the monomers, dimers and tetramers.

The current invention also unexpected found that for a larger protein such as a fusion protein containing an immunoglobulin domain, the dynamic binding capacity is higher for the monomer and dimmer as compared to the tetramer. Further, the highest relative capacity (i.e. capacity expressed as mg protein/mg ligand) was obtained for the monomers. Our study shows that the increases dynamic binding capacity in the monomers and dimers is not mainly an effect of ligand density, but probably due to higher utilization of available binding sites caused by reduced sterical hindrance (for the relatively bulky fusion protein) and/or faster kinetics.

It is understood that the term “immunoglobulin containing proteins” embraces antibodies and fusion proteins comprising an antibody portion as well as antibody fragments and mutated antibodies, as long as they have substantially maintained the binding properties of an antibody. The antibodies can be monoclonal antibodies or polyclonal antibodies. Preferably the antibodies are IgG, IgA and/or IgM, from a mammalian species, such as a human

In one embodiment, the invention relates to a matrix for affinity separation, which matrix comprises monomeric or dimeric ligands that comprise immunoglobulin-binding protein coupled to a solid support. Preferably, at least one asparagine residue of the protein has been mutated to an amino acid other than glutamine. The present matrix, when compared to a matrix comprised of the tetrameric ligand, exhibits an increased binding capacity. The mutated protein ligand is preferably an Fc-fragment-binding protein, and can be used for selective binding of IgG, IgA and/or IgM, preferably IgG.

The matrix according to the invention can comprise the mutant protein as described above in any embodiment thereof as ligand. In the most preferred embodiment, the ligands present on the solid support comprise a monomer as described above.

The solid support of the matrix according to the invention can be of any suitable well-known kind. A conventional affinity separation matrix is often of organic nature and based on polymers that expose a hydrophilic surface to the aqueous media used, i.e. expose hydroxy (—OH), carboxy (—COOH), carboxamido (—CONH₂, possibly in N— substituted forms), amino (—NH₂, possibly in substituted form), oligo- or polyethylenoxy groups on their external and, if present, also on internal surfaces. In one embodiment, the polymers may, for instance, be based on polysaccharides, such as dextran, starch, cellulose, pullulan, agarose etc, which advantageously have been cross-linked, for instance with bisepoxides, epihalohydrins, 1,2,3-trihalo substituted lower hydrocarbons, to provide a suitable porosity and rigidity. In the most preferred embodiment, the solid support is porous agarose beads. The supports used in the present invention can easily be prepared according to standard methods, such as inverse suspension gelation (S Hjertén: Biochim Biophys Acta 79(2), 393-398 (1964). Alternatively, the base matrices are commercially available products, such as SEPHAROSE™ FF (GE Healthcare). In an embodiment, which is especially advantageous for large-scale separations, the support has been adapted to increase its rigidity, and hence renders the matrix more suitable for high flow rates.

Alternatively, the solid support is based on synthetic polymers, such as polyvinyl alcohol, polyhydroxyalkyl acrylates, polyhydroxyalkyl methacrylates, polyacrylamides, polymethacrylamides etc. In case of hydrophobic polymers, such as matrices based on divinyl and monovinyl-substituted benzenes, the surface of the matrix is often hydrophilised to expose hydrophilic groups as defined above to a surrounding aqueous liquid. Such polymers are easily produced according to standard methods, see e.g. “Styrene based polymer supports developed by suspension polymerization” (R Arshady: Chimica e L'Industria 70(9), 70-75 (1988)). Alternatively, a commercially available product, such as Source™ (GE Healthcare) is used.

In another alternative, the solid support according to the invention comprises a support of inorganic nature, e.g. silica, zirconium oxide etc.

In yet another embodiment, the solid support is in another form such as a surface, a chip, capillaries, or a filter.

As regards the shape of the matrix according to the invention, in one embodiment the matrix is in the form of a porous monolith. In an alternative embodiment, the matrix is in beaded or particle form that can be porous or non-porous. Matrices in beaded or particle form can be used as a packed bed or in a suspended form. Suspended forms include those known as expanded beds and pure suspensions, in which the particles or beads are free to move. In case of monoliths, packed bed and expanded beds, the separation procedure commonly follows conventional chromatography with a concentration gradient. In case of pure suspension, batch-wise mode will be used.

The ligand may be attached to the support via conventional coupling techniques utilising, e.g. amino and/or carboxy groups present in the ligand. Bisepoxides, epichlorohydrin, CNBr, N-hydroxysuccinimide (NHS) etc are well-known coupling reagents. Between the support and the ligand, a molecule known as a spacer can be introduced, which will improve the availability of the ligand and facilitate the chemical coupling of the ligand to the support. Alternatively, the ligand may be attached to the support by non-covalent bonding, such as physical adsorption or biospecific adsorption.

In an advantageous embodiment, the present ligand has been coupled to the support by thioether bonds. Methods for performing such coupling are well-known in this field and easily performed by the skilled person in this field using standard techniques and equipment. In an advantageous embodiment, the ligand is firstly provided with a terminal cysteine residue for subsequent use in the coupling. The skilled person in this field also easily performs appropriate steps of purification.

In certain embodiments of the invention, the conditions for the adsorption step may be any conventionally used, appropriately adapted depending on the properties of the target antibody such as the pI thereof. The optional wash step can be performed using a buffer commonly used such as a PBS buffer.

The elution may be performed by using any commonly used buffer. In an advantageous embodiment, the recovery of antibodies is achieved, in a monomeric ligand system, by adding an eluent having a pH in the range of 4.0-4.4, preferably 4.2-4.4. In a similar embodiment, the recovery of antibodies is achieved, in a dimeric ligand system, by adding an eluent having a pH in the range of 3.8-4.2, preferably 3.9-4.0. Thus, an advantage of these embodiments is that the target antibody is exposed to pH values during elution which are as a rule higher than conventionally used with protein A-based ligands, which for most antibodies will result in lower risk of denaturation caused by reduced pH, less aggregates and increased yield of the target.

The present method is useful to capture target antibodies, such as a first step in a purification protocol of antibodies which are e.g. for therapeutic or diagnostic use. In one embodiment, at least 75% of the antibodies are recovered. In an advantageous embodiment, at least 80%, such as at least 90%, and preferably at least 95% of the antibodies are recovered using an eluent having a pH in the range of 3.8-4.2 for dimeric ligand systems and 4.0-4.4 for monomeric ligand systems. The present method may be followed by one or more additional steps, such as other chromatography steps. Thus, in a specific embodiment, more than about 98% of the antibodies are recovered.

As discussed earlier, for either SpA or protein Z ligand, when at least one asparagine residue is mutated to an amino acid other than glutamine or aspartic acid, affinity media including these mutant ligands can better withstand cleaning procedures using alkaline agents (US 2005/0143566). The increased stability means that the mutated protein's initial affinity for immunoglobulin is essentially retained for a prolonged period of time. Thus its binding capacity will decrease more slowly than that of the parental molecule in an alkaline environment. The environment can be defined as alkaline, meaning of an increased pH-value, for example above about 10, such as up to about 13 or 14, i.e. from 10-13 or 10-14, in general denoted alkaline conditions. Alternatively, the conditions can be defined by the concentration of NaOH, which can be up to about 1.0 M, such as 0.7 M or specifically about 0.5 M, accordingly within a range of 0.7-1.0 M.

Thus, the affinity to immunoglobulin i.e. the binding properties of the present ligand, and hence the capacity of the matrix, is not essentially changed in time by treatment with an alkaline agent. Conventionally, for a cleaning in place treatment of an affinity separation matrix, the alkaline agent used is NaOH and the concentration thereof is up to 0.75 M, such as 0.5 M. Thus, its binding capacity will decrease to less than about 70%, preferably less than about 50% and more preferably less than about 30%, such as about 28%, after treatment with 0.5 M NaOH for 7.5 h.

The increased chemical stability of the mutated protein according to the invention can easily be confirmed by the skilled person in this field e.g. by routine treatment with NaOH at a concentration of 0.5 M. In this context, it is to be understood that similar to what is said above, an “increased” stability means that the initial stability is retained during a longer period of time than what is achieved by the parental molecule.

In a further aspect, the present invention relates to a method of isolating an immunoglobulin, such as IgG, IgA and/or IgM, wherein a ligand monomer, a dimer or a matrix according to the invention is used. Thus, the invention encompasses a process of chromatography, wherein at least one target compound is separated from a liquid by adsorption to a ligand monomer, a dimer or matrix described above. The desired product can be the separated compound or the liquid. Thus, this aspect of the invention relates to affinity chromatography, which is a widely used and well-known separation technique. In brief, in a first step, a solution comprising the target compounds, preferably antibodies as mentioned above, is passed over a separation matrix under conditions allowing adsorption of the target compound to ligands present on said matrix. Such conditions are controlled e.g. by pH and/or salt concentration i.e. ionic strength in the solution. Care should be taken not to exceed the capacity of the matrix, i.e. the flow should be sufficiently slow to allow a satisfactory adsorption. In this step, other components of the solution will pass through in principle unimpeded. Optionally, the matrix is then washed, e.g. with an aqueous solution, in order to remove retained and/or loosely bound substances. The present matrix is most advantageously used with an intermediate washing step utilizing additives such as solvents, salts or detergens or mixture there of. In a next step, a second solution denoted an eluent is passed over the matrix under conditions that provide desorption i.e. release of the target compound. Such conditions are commonly provided by a change of the pH, the salt concentration i.e. ionic strength, hydrophobicity etc. Various elution schemes are known, such as gradient elution and step-wise elution. Elution can also be provided by a second solution comprising a competitive substance, which will replace the desired antibody on the matrix. For a general review of the principles of affinity chromatography, see e.g. Wilchek, M., and Chaiken, I. 2000. An overview of affinity chromatography. Methods Mol. Biol. 147: 1-6.

### EXAMPLES

Below, the present invention will be described by way of examples, which are provided for illustrative purposes only and accordingly are not to be construed as limiting the scope of the present invention as defined by the appended claims. All references given below and elsewhere in this application are hereby included herein by reference.

### Example 1

The aim of this study was to compare the performance of media prototypes based on agarose immobilized with monomers, dimers of tetramers of alkaline stabilized protein A, i.e. the SuRe ligand domain (below named z1, z2, and z4 respectively) by:
Determination of dynamic binding capacity for two different Monoclonal Antibodies expressed in CHO cell culture.
Comparison of elution pH obtained with the various ligands
Measuring clearance of host cell proteins at a sample load of 70% of dynamic binding capacity
at 10% breakthrough.

### 1. Experimental

### 1.1. Media Prototypes

| | |
|---|---|
| MabSelect SuRe: Lot 312257 | (ligand density 5.6 mg/ml) |
| HFA35 Z1: U1975095 | (ligand density 1.64 mg/ml) |
| HFA35 Z2: U1975098 | (ligand density 3.46 mg/ml) |

### 1.2. Chemicals and Samples

PBS buffer, SIGMA, P4417-100Tab

### NaOH, Merck, 1.06649.1000

Tri-sodium citrate dehydrate, Merck, 1.06448.1000
MAb 1, host cell clarified feed (HCCF), 1.1 mg MAb/ml
MAb 2, host cell clarified feed, 1.8 mg MAb/ml

### 1.3. Systems

ÄKTA Explorer 100, AL E100.
ÄKTA Explorer 10, HH E 10S.

### Spectrophotometer, UltroSpec 3000 pro.

### 2. Methods

### 2.1 Frontal Analysis

Frontal analysis was performed with two different host cell clarified feed containing MAbs (MAb 1 and MAb 2).

A preprogrammed Unicorn method, consisting of 6 blocks was used:

1. Equilibration with PBS buffer for 5 CV.

2. Loading of feed until approximately 80% breakthrough of the MAb.

3. Wash out of unbound material with PBS buffer for 10 CV.

4. Elution in a 20 CV gradient from 60 mM citrate pH 6.0 to 60 mM citrate pH 3.0.

5. CIP with 0.1 M NaOH at 0.3 ml/min for 15 minutes.

6. Re-equilibration with PBS.

UV absorbance at 295 nm was used for determination of breakthrough. Before frontal analysis, the feed was injected by-passing the column to obtain a maximum absorbance value corresponding to the MAb, host cell proteins (HCP) and other components in the feed. The “plateau flow through” during early sample application (corresponding to components in the feed that do not bind to the column) was subtracted from the UV-curve, and the resulting absorbance values was used for calculation of dynamic binding capacity at 5, 10 and 80% breakthrough according to equation 1.

Q_{BX%}=(V_{x%}−V₀)C₀/V_{c} Equation 1

where V_{x%}=applied volume of sample at x % breakthrough, C₀=sample concentration (mg/ml), V_{c}=geometric total volume and V₀=void volume.

### 2.2 HCP Clearance

Sample was applied to a final load 70% of the Q_{B10%} value. After wash out of unbound material, elution was performed with 60 mM citrate pH 3.5. Eluted fractions were pooled and diluted with one 1/10 of 0.2 M sodium phosphate, 1% BSA, 0.5% tween pH 8.0. The samples were then analyzed for HCP content using Gyros methodology. The absorbances in the supernatants were measured at 280 nm. The Lambert-Beer law (Eq. 2) was used for calculation of MAb concentration. HCP concentration in ppm was then obtained by dividing HCP concentration in ng/ml by MAb concentration in mg/ml.

A=C·l·ε Equation 2

where C=protein concentration (mg/mL), A=absorbance at 280 nm, l=path length (cm)=1 and ε=extinction coefficient (mg mL⁻¹ cm⁻¹)=1.7.

Yield was calculated according to equation 3:

Yield (%)=100·(Vₚₒₒₗ·Cₚₒₒₗ)/(Vᵢₙ·C₀) Equation 3

where Vₚₒₒₗ=volume of pooled fractions, Cₚₒₒₗ=MAb concentration in pool, Vᵢₙ=volume of sample loaded onto the column and C₀=sample concentration (mg/ml).

### 2.3 Determination of Purity by Analytical Size Exclusion Chromatography.

Analytical size exclusion chromatography, on SUPERDEX 200 5/150 GL, was performed by standard methods according to manual. The elution buffer was PBS pH 7.4 (SIGMA) and the sample volume 254 Purity of the MAbs was determined by integration of the chromatograms.

### 3. Results and Discussion

### 3.1 Frontal Analysis

### 3.1.1 MAb 1

The dynamic binding capacity (DBC) was calculated for 5, 10 and 80% breakthrough. Results are shown in Table 1a. DBC at 5 and 10% breakthrough was equivalent for MabSelect SuRe (z4) and z2, while the values were lower for z1. However as shown in Table 1b, the highest relative capacity (i.e. capacity expressed as mg MAb/mg ligand) was obtained for ligands with fewer z-units, i.e. z1>z2>z4.

A comparison of Q_{B10%}/Q_{B80%} was used as a measure of the kinetics (higher value indicates faster kinetics). The results show that somewhat slower kinetic is obtained for z4 compared to z1 or z2.

Elution in a pH gradient gave approximately the same elution pH for z4 and z2 (pH 3.6-3.7) while the MAb eluted at somewhat higher pH from z1 (pH 4.0).

DBC was also determined at different residence times between 1 and 2.4 minutes. As expected DBC decreases at lower residence time (i.e. higher flow velocity). However the decrease in DBC is lower for z2 compared to z4, and further improvement is obtained with z1 (Table 1a). Thus for lowest drop in DBC, the monomeric ligand is preferable.

TABLE 1a
Summary of results from frontal analysis with MAb 1.

| MAb 1 | RT 2.4 min | | | | Elution at |
|---|---|---|---|---|---|
| Ligand | QB 5% | QB 10% | QB 80% | QB10/80 | pH |
| Z4* | 27 | 29 | 51 | 0.6 | 3.6 |
| Z2 | 28 | 29 | 39 | 0.75 | 3.7 |
| Z1 | 18 | 19 | 25 | 0.75 | 4 |

| | |
|---|---|
| RT 1.5 min | RT 1.0 min |

| | QB 5% | QB 10% | QB 5% | QB 10% |
|---|---|---|---|---|
| Z4* | ~21 | 23 | 15 | 16 |
| Z2 | 23 | 25 | ND | ND |
| Z1 | 16 | 17 | 14 | 16 |

*Z4 = MabSelect SuRe

TABLE 1b
Results from frontal analysis with MAb
1, expressed as relative capacity.

| MAb 1 | Relative capacity mg Mab/mg ligand | | |
|---|---|---|---|
| Ligand | QB 5% | QB 10% | QB 80% |
| Z4 | 4.8 | 5.2 | 9.1 |
| Z2 | 8.1 | 8.4 | 11.3 |
| Z1 | 11.0 | 11.6 | 15.2 |

(Residence time: 2.4 min.)

### 3.1.2 MAb 2

Frontal analysis was performed at a residence time of 2.4 minutes, and the dynamic binding capacity (DBC) was calculated for 5, 10 and 80% breakthrough. Results are shown in Table 2a. As for MAb 1, DBC at 5 and 10% breakthrough was almost equivalent for MabSelect SuRe (z4) and z2, while a lower value was obtained for z1. As above, the highest relative capacity was obtained for ligands with fewer z-units, i.e. z1>z2>z4. A comparison of Q_{B10%}/Q_{B80%} shows that faster kinetics is obtained for ligands with fewer z-units, i.e. z4<z2<z1.

Elution in a pH gradient gave slightly higher elution-pH for z1 compared to z2 (no value was obtained for z4, due to an experimental error).

TABLE 2a
Summary of results from frontal analysis with MAb 2.

| | RT 2.4 min | | | | Elution at |
|---|---|---|---|---|---|
| MAb 2 | QB 5% | QB 10% | QB 80% | QB10/80 | pH |
| Z4* | 29 | 32 | 59 | 0.53 | ND |
| Z2 | 32 | 33 | 49 | 0.67 | 3.8 |
| Z1 | 22 | 23 | 33 | 0.71 | 4 |

*Z4 = MabSelect SuRe

TABLE 2b
Results from frontal analysis with MAb
2, expressed as relative capacity.

| MAb 2 | Relative capacity | | |
|---|---|---|---|
| Ligand | QB 5% | QB 10% | QB 80% |
| Z4 | 5.2 | 5.7 | 10.5 |
| Z2 | 9.2 | 9.5 | 14.2 |
| Z1 | 13.4 | 14.0 | 20.1 |

(Residence time: 2.4 min.)

### 3.2 HCP Clearance

Sample was applied to a final load of 70% of the Q_{B10%} value, and the elution pools were analyzed for HCP content and by analytical size exclusion chromatography on Superdex 5/150 GL. The results obtained for purification of MAb 1 and 2 are shown in Table 3. Clearance of HCP was almost equivalent for z1, z2 and z4. However, the relative reduction of HCP (i.e. Cₛₜₐᵣₜ material/Cₚₒₒₗ) and percentage yields were higher for MAb1 than for MAb2.

Elution pools for both MAbs contained 4.4-6.8% dimer/aggregates as determined by analytical size exclusion chromatography. No clear trend could be observed for the different ligands. Elution of MAb 2 at higher pH (i.e. 3.75 instead of 3.5) from z2-media resulted in lower yield, somewhat higher reduction of HCP and dimer/aggregates.

TABLE 3
Summary of results from purification of MAb 1 and 2.

| | Yield | mg | ng | ppm | D/A | Purity |
|---|---|---|---|---|---|---|
| MAb 1 | (%) | Mab/ml | HCP/ml | HCP | %² | %³ |
| Start material | | 1.1 | 36500 | 36869 | | |
| Z4¹ | 95 | 7.5 | 1090 | 161 | 4.4 | 95 |
| Z2 | 97 | 7.6 | 1100 | 161 | 5.7 | 94 |
| Z1 | 98 | 6.1 | 1130 | 206 | 5.5 | 92 |
| | Yield | mg | ng | ppm | D/A | Purity |
| MAb 2 | (%) | Mab/ml | HCP/ml | HCP⁴ | %² | %³ |
| Start material | | 1.8 | 19000 | 11728 | | |
| Z4¹ | 83 | 8.8 | 2350 | 297 | 4.7 | 94 |
| Z2 | 92 | 10.7 | 4450 | 462 | 6.8 | 93 |
| Z2 (elution | 85 | 7.95 | 2050 | 287 | 3.2 | 96 |
| pH 3.75) | | | | | | |
| Z1 | 78 | 8 | 1900 | 264 | 4.4 | 95 |

¹Z4 = MabSelect SuRe.
²D/A = aggregates and dimers as determined by analytical size exclusion chromatography.
³Purity = (peak area of monomeric MAb)/(total peak area), as determined by integration of chromatogram from analytical size exclusion chromatography.

### 4. Conclusions

The highest dynamic binding capacity was obtained for z4 and z2. The lowest capacity was obtained for z1 that also has the lowest ligand density (1.64 mg/ml). However, the highest relative capacity (mg MAb/mg ligand) was obtained for ligands with fewer z-units.

Faster kinetics and lower decrease in dynamic binding capacity at decreased residence time are obtained for the agarose based media prototypes coupled with ligands with fewer z-units. Thus, the capacity drop at shorter residence time was lowest on z1.

The elution pH for both MAbs was somewhat higher on z 1. Higher elution pH can be an advantage for MAbs susceptible to aggregation at low pH.

Clearance of HCP was almost equivalent for z1, z2 and z4.

### Example 2

The aim of this study was to compare the performance of media prototypes based on agarose based media, coupled with different version of alkaline stabilized protein A, i.e. monomer, dimer and tetramer of the Z domain (below named Z1, Z2, Z4) by
Determination of dynamic binding capacity for “MAb 3”
Measuring clearance of host cell proteins at a sample load of 70% of dynamic binding capacity at 10% breakthrough.

### 1. Experimental

### 1.1 Chromatography Media and Filters

| | |
|---|---|
| HFA35 Z4: MabSelect SuRe batch 10007589 | (ligand density 5.9 mg/ml) |
| HFA35 Z1: U1975095 | (ligand density 1.64 mg/ml) |
| HFA35 Z2: U1975098 | (ligand density 3.46 mg/ml) |

### Superdex 200 5/150 GL, GE Healthcare, 28-9065-63

### 1.2. Chemicals

PBS buffer, SIGMA, P4417-100Tab

### NaCl, MERCK, 1.06404.1000

### NaOH, MERCK, 1.06649.1000

Citric acid, MERCK, 1.00244.0500

### Aceton, MERCK, 1.00014.2511

Raw dextran, GE Healthcare (no lot number)
Preservation solution for dilution of samples for ELISA:
0.2 M sodium phosphate, 1% BSA, 0.5% tween pH 8.
MAb 3 [VH3] in host cell clarified feed (HCCF), 3 mg/ml (extinction coefficient 1.4)

### 1.3. Systems

### See Example 1.

### 2. Methods

### 2.1. Frontal Analysis

Frontal analysis was performed with MAb 3. A preprogrammed Unicorn method, consisting of 6 blocks was used:

1. Equilibration with PBS buffer for 5 CV.

2. Loading of feed until >10% breakthrough of the MAb.

3. Wash out of unbound material with equilibration buffer for 10 CV.

4. Elution with 60 mM citrate pH 3.5 for 6 CV.

5. CIP with 0.1 M NaOH at 0.3 ml/min for 15 minutes.

6. Re-equilibration with equilibration buffer.

The UV absorbance at 295 nm was used for determination of breakthrough essentially as described in Example 1. Dynamic binding capacity at 10% breakthrough was calculated according to Equation 1.

### 2.2. HCP-Clearance

Sample was applied to a final load of 70% of the Q_{B10%} value. After wash out of unbound material, elution was performed with 0.1 M citrate pH 3.3. Eluted fractions were pooled and diluted with 1 tenth of preservation solution. The samples were then analyzed for HCP content, and for aggregate content by analytical size exclusion chromatography according to 2.3. The absorbances in the supernatants were measured at 280 nm. The Lambert-Beer law (Equation 2) was used for calculation of MAb concentration (c=extinction coefficient (mg mL⁻¹ cm⁻¹)=1.4). HCP in ppm was then obtained by dividing HCP concentration in ng/ml by MAb concentration in mg/ml.

Antibody yield was calculated according to equation 3.

### 2.3. Determination of Purity by Analytical Size Exclusion Chromatography

### See Example 1.

### 3. Results and Discussion

### 3.1 Frontal Analysis

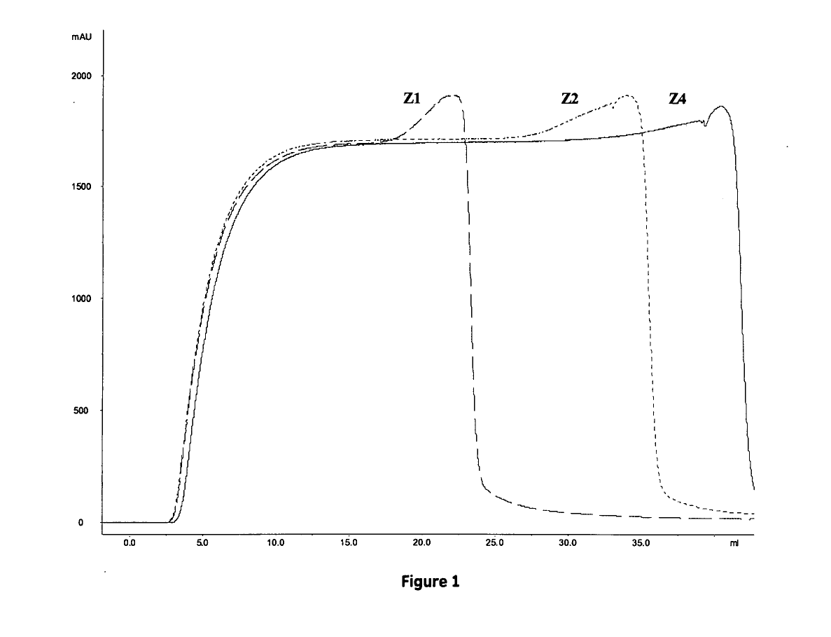

The dynamic binding capacity (DBC) at various residence times (1, 2.4 and 6 minutes) was calculated at 10% breakthrough. Results are shown in Table 4 and FIG. 1. The highest DBC was obtained for Z4 (i.e. MabSelect SuRe) followed by Z2. The lowest DBC was obtained for Z1. However, as shown in Table 5, the highest relative capacity (i.e mg MAb/ml ligand) was obtained with ligands with fewer Z-units, Z1>Z2>Z4. This result is in accordance with previously results obtained in Example 1.

TABLE 4
Summary of results from frontal analysis.

| DBC 10% (mg/ml) | Ligand | | |
|---|---|---|---|
| Ligand density (mg/ml) | 1.64 | 3.46 | 5.9 |
| Residence time | Z1 | Z2 | Z4 |
| 1 | 17 | 23 | 21 |
| 2.4 | 21 | 32 | 35 |
| 6 | 23 | 38 | 43 |

TABLE 5
Results from frontal analysis expressed as relative capacity.

| | Residence time (min) | | | Ligand density |
|---|---|---|---|---|
| (mg/mg ligand) | 1¹ | 2.4 | 6 | mg/ml resin |
| Z1 | 10 | 13 | 14 | 1.64 |
| Z2 | 7 | 9 | 11 | 3.46 |
| Z4 | 4 | 6 | 7 | 5.9 |

¹DBC at 1 minute residence time was difficult to determine due to leakage of MAb during sample application.

### 3.2. HCP Clearance, Yield and Purity

Sample was applied, at a residence time of 2.4 min, to a final load of 70% of DBC at 10% breakthrough, and the elution pools were analyzed for HCP content and by analytical size exclusion chromatography on Superdex 200 5/150 GL. The results are shown in Table 6. Clearance of HCP, yield and levels of high- and low molecular weight material was almost equivalent for Z1, Z2 and Z4.

As a control experiment, the start material and the flow-through fraction from HFA35 Z1 were analyzed by analytical size exclusion chromatography on Superdex 200 5/150 GL. Almost no MAb could be detected in the flow through pool.

TABLE 6
Summary of results from purification of MAb 3

| | | | | | Pu- | HMW/ |
|---|---|---|---|---|---|---|
| | Yield | mg | HCP | HCP | rity of | LMW |
| Sample | (%) | Mab/ml | (ng/ml)¹ | (ppm)¹ | MAb (%)² | (%)³ |
| Start material | | 3 | 22500 | 7500 | | |
| Z1 | 100 | 5.5 | 40 | 7 | 90.8 | 4.3/4.6 |
| Z2 | 99 | 9.1 | 145 | 16 | 81 | 4.4/5.1 |
| Z4 | 99 | 14 | 220 | 16 | 91.7 | 5.2/3.0 |

²Purity of the MAb was determined by analytical size exclusion chromatography on Superdex 200 5/150 GL.

### Example 3

Fc-binding protein is a recombinant-DNA drug made by combining two proteins (a fusion protein). It links human soluble TNFα receptor to the Fc component of human immunoglobulin G1 (IgG₁). It is a large molecule, with a molecular weight of 150 kDa., that binds to TNFα and decreases its role in disorders involving excess inflammation in humans and other animals, including autoimmune diseases such as ankylosing spondylitis, juvenile rheumatoid arthritis, psoriasis, psoriatic arthritis, rheumatoid arthritis, and, potentially, in a variety of other disorders mediated by excess TNFα. This therapeutic potential is based on the fact that TNF-alpha is the “master regulator” of the inflammatory response in many organ systems.

The aim of this study was to compare the performance of media prototypes based on HFA 35, coupled with different version of alkaline stabilized protein A, i.e. monomer, dimer and tetramer of the Z domain (below named Z1, Z2, Z4) by determination of dynamic binding capacity for Fc-fusion protein expressed in clarified CHO cell culture supernatant.

### 1. Experimental

### 1.1. Chromatography Media and Filters

### HiTrap MabSelect SuRe, GE Healthcare, lot 10021346

Pre-filters: 5 μm polypropylene filter, Millipore, AN5004700
For other media see Example 2.

### 1.2. Chemicals

### TRIS, MERCK, 1.08382.0500

PBS buffer, SIGMA, P4417-100Tab

### NaCl, MERCK, 1.06404.1000

### NaOH, MERCK, 1.06649.1000

### NaN₃, MERCK, K2806788

### HCl, MERCK, 1.09973.001

Acetic acid, MERCK, 1.00063.2511

### Aceton, MERCK, 1.00014.2511

Raw dextran, GE Healthcare (no lot number)
Feed containing the fusion protein Fc-Fusion Protein from CHO cell culture supernatant. The feed contained 0.48 mg Fc-Fusion Protein 1/ml.

### Purified Fc-Fusion Protein

### MAb 3

### 1.3. Systems

ÄKTA Explorer 100, AL E100.
ÄKTA Explorer 100, AL L100.
ÄKTA Explorer 10. LK-E 10XT

### 2. Methods

### 2.1. Sample Preparation

CHO cell culture supernatant was filtered through 5 μm pre-filter. NaN₃ was added to a final concentration of 0.05% (w/v). The sample was stored in a refrigerator at approximately 6° C.

### 2.2. Frontal Analysis

Frontal analysis was performed with Fc-Fusion Protein feed. A preprogrammed Unicorn method, consisting of 6 blocks was used:
1. Equilibration with 25 mM TRIS-HCl, 0.15 M NaCl pH 7.4 for 5 CV.
2. Loading of feed until >10% breakthrough of the fusion protein (Note that the breakthrough could not be monitored by the UV-signal, due to the low feed concentration (0.48 mg T2/ml). 1.3 ml fractions were collected and analyzed as described below).
3. Wash out of unbound material with equilibration buffer for 10 CV.
4. Elution with 0.1 M acetic acid pH 3.6 for 6 CV.
5. CIP with 0.5 M NaOH at 0.4 ml/min for 15 minutes
   Selected fractions during sample loading were analyzed on a HiTrap MabSelect SuRe column using the same buffers as above. The elution peaks were integrated, and the peak areas in mAUxml were compared to the peak area for the start material. The volume applied at 10% breakthrough (V_{10%}) was defined out from the fraction with 10% of the peak area in the start material. The dynamic binding capacity (Q_{B10%}) was then calculated according to Equation 1.

### 2.3 Size Exclusion Chromatography

Analytical size exclusion chromatography was performed as in Example 1.

The retention volume (V_{R}) for each sample was determined at the peak apex. The void volume (V₀) was determined with ˜5% raw dextran and the total liquid volume (Vₜ) with 5% acetone in PBS-buffer.

The distribution coefficient, K_{D}, represents the fraction of the stationary phase which is available for diffusion of a given solute species (Gel filtration Principles and Methods (Pharmacia LKB Biotechnology 1991); Lars Hagel, Gel Filtration, in Protein Purification, Principles, High Resolution Methods and Applications (Eds J-C Jansson and L Ryde'n, VHS Publishers, New York, 1989., Ch. 3.)). K_{D} was calculated from Equation 4:

K_{D}=(V_{R}−V₀)/Vp=(V_{R}−V₀)/(Vₜ−V₀)

where Vp=pore volume (=Vₜ−V₀).

### 3. Results and Discussion

The dynamic binding capacity (DBC) for Fc-Fusion protein at 10% breakthrough was determined at two different residence times (i.e. 2.4 and 6 minutes). Results are shown in Table 7. In contrast to earlier results with MAbs, DBC for Fc-Fusion Protein is higher for Z1 and Z2 compared to Z4.

TABLE 7
Summary of results from frontal analysis with Fc-Fusion Protein.

| | |
|---|---|
| DBC10% | Ligand |

| Residence time | Z1 | Z2 | Z4 | Z4 (low) |
|---|---|---|---|---|
| 2.4 min | 10 | 8 | 6 | 5 |
| 6 min | 12 | 14 | 10 | 9 |

Even though Z1 has lower ligand density than Z4 (1.65 and 5.9 mg/ml respectively), DBC for Z1 is higher, especially at the lower residence time. Also Z2 (ligand density 3.46 mg/ml) has higher DBC than Z4, especially at higher residence time. The highest relative capacity (i.e. capacity expressed as mg Fc-Fusion Protein/mg ligand) was obtained for Z1 (Table 8).

TABLE 8
Results from frontal analysis expressed as relative capacity.

| | | |
|---|---|---|
| Relative capacity | Residence time (min) | Ligand density |

| (mg/mg ligand) | 2.4 | 6 | mg/ml |
|---|---|---|---|
| Z1 | 6.1 | 7.3 | 1.64 |
| Z2 | 2.3 | 4.0 | 3.46 |
| Z4 | 1.0 | 1.7 | 5.9 |

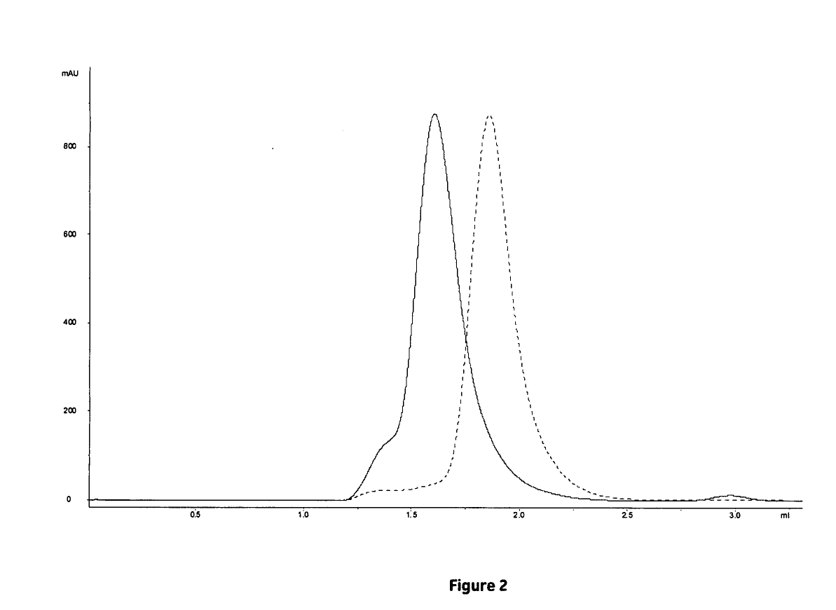

Analytical size exclusion chromatography on Superdex 200 5/150 GL showed that Fc-Fusion Protein elutes earlier than MAb 3 (FIG. 2). The distribution coefficient (K_{D}) for Fc-Fusion Protein and MAb 3 was calculated to 0.28 and 0.43 respectively. Thus, even though the difference in molecular weight is small, Fc-Fusion Protein behaves like a much bulkier molecule (i.e. higher solute radious) than MAb.

### Example 4

The aim of this study was to investigate elution pH of several monoclonal antibodies purified on different z-ligands on agarose based matrix. Elution was performed in a linear pH gradient. Elution pH was measured at peak maximum. The samples purified on the monomer ligand prototype eluted with slightly higher pH compared with the other prototypes. The results also showed that elution pH depends on ligand density. Higher sample load was investigated with polyclonal IgG. No indications were seen that higher sample load affect the elution pH. Elution conditions can be a benefit for further optimization of each MAb. MAb is captured at neutral pH and eluted by acidic pH. If MAb's can be eluted with increased pH then this can be an advantage for Mabs susceptible to aggregation at low pH. Higher elution pH can prevent aggregation and give higher recovery.

In this example we investigated elution pH of seven different immunoglobulin molecules on different alkaline stabilized z-ligands of the MabSelect SuRe ligand domain. We also investigated the effect of high load of polyclonal IgG on various z-ligands.

### 1. Experimental

### 1.1. Materials/Investigated Units

### 1.1.1. Columns

Agarose based media with various Z ligands:

| | |
|---|---|
| Agarose based media Z1: U1975077 | (1.98 mg/ml) |
| Agarose based media Z2: U1975098 | (3.46 mg/ml) |
| MabSelect SuRe (Z4) lot: 312257 | (5.6 mg/ml) |
| MabSelect SuRe (Z4) lot: 306928 | (5.6 mg/ml) |

### Binding Capacity:

TABLE 9
QB₁₀ data with polyclonal Ig G (gammanorm).

| | Ligand density. | QB10 |
|---|---|---|
| ID | (mg/ml) | (mg/ml gel) |
| Agarose based media Z1: U1975077 | 1.98 | 23.1 |
| Agarose based media Z2: U1975098 | 3.46 | 37.1 |
| MabSelect SuRe (Z4) lot: 312257 | 5.6 | 37.5* |
| MabSelect SuRe (Z4) lot: 306928 | 5.6 | 33.1 |
| MabSelect SuRe (Z4) lot: 10007489 | 5.9 | 34.68 |

### 1.1.2. Buffers

10 mM phosphate buffer (PBS, SIGMA, P4417-100Tab)
60 mM Na-citrate pH 6 (Na-citrate M. 294.1 g/mol) pH adjustment with HCl
60 mM Na-citrate pH 3 (Na-citrate M. 294.1 g/mol) pH adjustment with HCl

### 0.3 M NaOH

### 1.1.3. Samples

All samples were pure fractions. PD-10 column was used for buffer exchange.
Polyclonal human IgG (Octapharma, gammanorm)

### Mab1

### Mab 2

Fc-fusion protein, lot: 1510
CHO-cells supernatant containing IgG
MAb4 (purified on HiTrap MabSelect SuRe)

### MAb 3

0.5-1.5 mg sample was loaded/ml media.

### 1.2. Methods

UNICORN method

### Equilibration: 5 CV

Sample volume: 1 ml (low load)
Wash out unbound sample: 5 CV

### Elution: Gradient pH 6 to pH 3 20 CV

### Re-equilibration: 5 CV

Residence time: 2.4 min
Elution was performed in a linear pH gradient. Elution pH was measured at peak maximum.

### 1.3. Equipment

| | |
|---|---|
| ÄKTAexplorer 10 | TF_E10 |
| Spectrophotometer | Molecular Devices Spextramax PLUS |
| Mettler Toledo | Seven Easy |

### 2. Results

Seven MAbs and Fc-binding proteins were purified on different z-ligand prototypes i.e. monomer (Z1), dimer (Z2) and SuRe (z4) with different ligand density. pH values of Fc-Fusion Protein and Mab 1 eluted on prototype SuRe (1.37 mg/ml) differ from the other samples on this column. The explanation might be that these two samples had been frozen/thawn before application.

MAB 3 and Mab 4 sample were purified on MabSelect media (rProtein A). The elution pH for MAB 3 was as expected lower on MabSelect than on the other z-prototypes. MAB 3 contains of a VH3 part, both the Fc part and the VH3 part bind to MabSelect media and a lower pH is needed for elution.

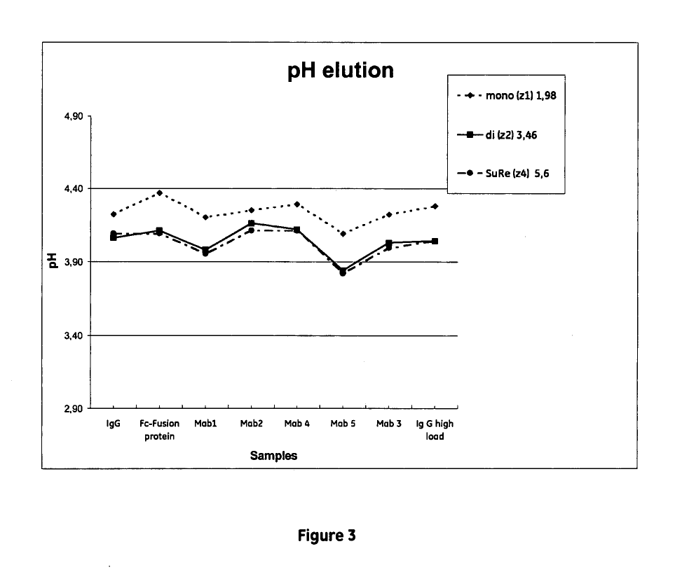

The results showed a dependency of elution pH and ligand density. When the samples were purified on the monomer ligand prototype they eluted with slightly higher pH compared with the other prototypes (FIG. 3).

FIG. 3 shows elution pH of different MAbs and Fc-fusion protein, low load, applied on various z-prototypes. The results confirm that elution pH is dependent on ligand density. The samples purified on the monomer ligand prototype eluted with slightly higher pH compared with the other prototypes. Higher sample load was investigated with polyclonal IgG. No indications were seen that higher sample load affect the elution pH.

As higher elution pH is needed to elute MAb's from monomer ligand prototype, this provides a benefit to avoid aggregates during the elution step. This and other characteristics make the monomer a good ligand for affinity chromatography.

The above examples illustrate specific aspects of the present invention and are not intended to limit the scope thereof in any respect and should not be so construed. Those skilled in the art having the benefit of the teachings of the present invention as set forth above, can effect numerous modifications thereto. These modifications are to be construed as being encompassed within the scope of the present invention as set forth in the appended claims.

What is claimed is:

1. In a method of separating one or more immunoglobulin containing proteins from a liquid, which method comprises
(a) contacting the liquid with a separation matrix comprising ligands immobilised to a support;
(b) allowing said immunoglobulin containing proteins to adsorb to the matrix by interaction with the ligands;
(c) optionally washing the adsorbed immunoglobulin containing proteins; and
(d) recovering said immunoglobulin containing proteins by contacting the matrix with an eluent which releases the proteins;
the improvement comprising that each of said ligands consists essentially of a monomer or a dimer of domain B of staphylococcal Protein A (SpA), protein Z, or a functional variant thereof.

2. (canceled)

3. The method of claim 1, wherein the ligands are alkali-stable by mutating at least one asparagine residue to an amino acid other than glutamine.

4. The method of claim 3, wherein the ligands have affinity for the Fc part of an immunoglobulin but lack affinity for the Fab part of an immunoglobulin.

5. The method of claim 4, wherein at least one glycine of said ligands has been replaced by an alanine.

6. The method of claim 4, wherein a glycine residue at position 29 has been changed to an alanine.

7. The method of claim 1, wherein the ligand is Protein Z in which the alkali-stability has been achieved by mutating at least one asparagine residue to an amino acid other than glutamine.

8. The method of claim 7, wherein the alkali-stability of Protein Z has been achieved by mutating at least the asparagine residue at position 23 to an amino acid other than glutamine.

9. The method of claim 1, wherein the recovery of immunoglobulin containing proteins is achieved by adding an eluent having a pH of 3.8-4.2.

10. The method of claim 1, wherein at least 80%, such as at least 90%, and preferably at least 95% of the immunoglobulin containing proteins are recovered using an eluent having a pH of 3.8-4.2.

11. The method of claim 1, wherein the recovery of immunoglobulin containing proteins is achieved by adding an eluent having a pH of 4.0-4.4.

12. The method of claim 1, wherein at least 80%, such as at least 90%, and preferably at least 95% of the immunoglobulin containing proteins are recovered using an eluent having a pH of 4.0-4.4.

13. The method of claim 1, wherein the immunoglobulin containing protein is a monoclonal antibody.

14. The method of claim 1, wherein the immunoglobulin containing protein is a polyclonal antibody.

15. The method of claim 1, wherein the immunoglobulin containing is a fusion protein containing an immunoglobulin containing fused with another protein.
